(19) United States
(12) Patent Application Publication
(10) Pub. No.: US 2014/0073888 A1
(43) Pub. Date: Mar. 13, 2014

(54) NON-INVASIVE METHOD FOR MONITORING AUTOREGULATION

(71) Applicants: Rakesh Sethi, Vancouver (CA); James N. Watson, Dunfermline (GB); Paul S. Addison, Edinburgh (GB)

(72) Inventors: Rakesh Sethi, Vancouver (CA); James N. Watson, Dunfermline (GB); Paul S. Addison, Edinburgh (GB)

(73) Assignee: NELLCOR PURITAN BENNETT LLC, Boulder, CO (US)

(21) Appl. No.: 13/606,312

(22) Filed: Sep. 7, 2012

(51) Int. Cl. A61B 5/0205; A61B 5/1455

(52) U.S. Cl. 600/324

(57) ABSTRACT

A system includes a controller that receives a blood pressure signal and an oxygen saturation signal. The blood pressure signal represents a non-invasive measure of blood pressure. The oxygen saturation signal represents a non-invasive measure of oxygen saturation. The controller generates an autoregulation status signal representing a status of cerebral autoregulation. The autoregulation status signal is based, at least in part, on a relationship between the measured blood pressure and the measured oxygen saturation. An exemplary method may include receiving the blood pressure signal and the oxygen saturation signal, defining a relationship between the measured blood pressure and the measured oxygen saturation, determining an autoregulation status based at least in part on the defined relationship, and generating an autoregulation status signal representing the determined autoregulation status.

### BACKGROUND

Cerebral blood flowsupplies oxygen and nutrients to the brain. A drop in blood flow can cause ischemia which may result in tissue damage or death of brain cells. An increase in blood flow can cause hyperminia which may result in swelling of the brain or edema. Autoregulation is a process that attempts to maintain an optimal blood flow to the brain.

Following a reduction in blood flow to the brain, the initial response of the body is peripheral vasoconstriction, which reduces blood flow to non-essential areas of the body while maintaining blood pressure. The secondary response is pressure autoregulation in the cerebral area. During autoregulation, cerebral arterioles dilate as cerebral pressure falls in the attempt to maintain blood flow. As cerebral pressure increases, cerebral arterioles constrict to reduce the blood flow that could also cause injuries.

### BRIEF DESCRIPTION OF THE DRAWINGS

FIG. 1 illustrates an exemplary system for non-invasively detecting cerebral autoregulation impairment.

FIG. 2 is a flowchart of an exemplary process that may be used by the system of FIG. 1.

FIG. 3 illustrates a chart of exemplary oxygen saturation and blood pressure values having a linear correlation that indicates properly functioning cerebral autoregulation.

### DETAILED DESCRIPTION

An exemplary system includes a controller that receives a blood pressure signal and an oxygen saturation signal. The blood pressure signal represents a non-invasive measure of blood pressure. The oxygen saturation signal represents a non-invasive measure of oxygen saturation. The controller generates an autoregulation status signal representing a status of cerebral autoregulation. The autoregulation status signal is based, at least in part, on a relationship between the measured blood pressure and the measured oxygen saturation

The controller may implement various processes to determine the autoregulation status of the patient. One exemplary process may include the following: receiving the blood pressure signal and the oxygen saturation signal, defining a relationship between the measured blood pressure and the measured oxygen saturation, determining an autoregulation status based at least in part on the defined relationship, and generating an autoregulation status signal representing the determined autoregulation status.

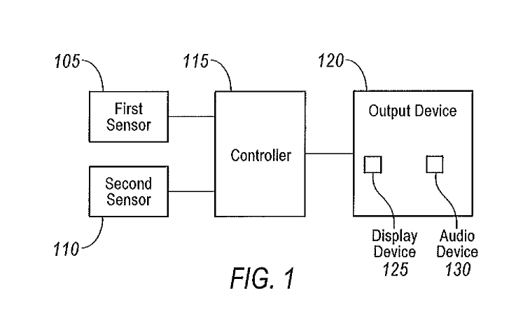

FIG. 1 illustrates an exemplary system 100 for detecting a status of cerebral autoregulation. As illustrated in FIG. 1, the system 100 includes a first sensor 105, a second sensor 110, a controller 115, and an output device 120. The system 100 may take many different forms and include multiple and/or alternate components and facilities. While an exemplary system 100 is shown, the exemplary components illustrated in Figure are not intended to be limiting. Indeed, additional or alternative components and/or implementations may be used.

The first sensor 105 and second sensor 110 may each include any device configured to non-invasively measure a physiological parameter of a patient. Example physiological parameters include blood pressure, regional oxygen saturation, or hemoglobin. In some exemplary approaches, the first sensor 105, the second sensor 110, or both, may include a near-infrared spectroscopy sensor configured to generate light in the near-infrared spectrum, from about 800 nm to about 2500 nm, and receive reflected light.

The first and second sensors 105 and 110 may in some circumstances process the reflected light at least to generate a representative signal of the measured physiological parameter. The representative signal, therefore, may indicate an amount of oxygen or hemoglobin in a patient's blood or a particular organ or other tissue. The representative signal may, in some instances, represent the patient's blood pressure. Blood pressure may be defined as the pressure exerted on blood vessel walls, such as arterial or venous walls, during each heartbeat. The blood pressure may include a systolic value, which represents the patient's maximum blood pressure, and a diastolic value, which represents the patient's minimum blood pressure. The blood pressure signal generated by the sensor may represent blood pressure values at various times.

In operation, the first sensor 105 and the second sensor 110 may each be placed on the same or different parts of the patient's body. Indeed, the first and second sensors 105 and 110 may in some instances be part of the same sensor as opposed to separate devices. The first sensor 105 may be configured to generate a blood pressure signal that represents the non-invasive blood pressure measurement while the second sensor 110 may be configured to generate an oxygen saturation, which may represent a non-invasive measurement of regional oxygen saturation (RSO₂) or hemoglobin (SpO₂).

The first and second sensors 105 and 110 may, in one possible approach, be part of an integrated oximetry system capable of non-invasively measuring blood pressure and oxygen saturation. The first and second sensors 105 and 110 may both transmit signals directly to a single computing device, such as the controller 115 discussed below, and may possibly be part of the same device as one another. One or both of the first and second sensors 105 and 110 may be further configured to measure other parameters beyond blood pressure and oxygen saturation, respectively. In some instances, the first and second sensors 105 and 110 may both be configured to measure blood pressure, regional oxygen saturation, hemoglobin, respiratory rate, respiratory effort, heart rate, saturation pattern detection, response to stimulus such as bispectral index (BIS) or electromyography (EMG) response to electrical stimulus, etc.

The controller 115 may include any device configured to receive and process representative signals, such as the blood pressure signal and the oxygen saturation signal, from the first sensor 105 and second sensor 110, respectively. The controller 115 may be further configured to determine, from the blood pressure signal and the oxygen saturation signal, whether cerebral autoregulation of the patient is impaired. The controller 115 may be configured to generate an autoregulation status signal that represents the autoregulation status of the patient. As described in greater detail below, the autoregulation status signal may be based at least in part on the measured blood pressure and measured oxygen saturation.

One way for the controller 115 to determine the cerebral autoregulation status of the patient is to derive a cerebral oximetry index measurement for the patient. The cerebral oximetry index (COx) is an index of vascular reactivity. Vascular reactivity is a property of blood vessels that allows for proper blood flow throughout the body through various mechanisms such as vasoconstriction (a narrowing of the blood vessel) and vasodilation (expansion of the blood vessel). The cerebral oximetry index measurement, therefore, relates to the vascular reactivity of the blood vessels within the brain.

The cerebral oximetry index measurement can be derived by the controller 115 based, at least in part, on the blood pressure signal and the oxygen saturation signal. For example, in one possible implementation, the controller 115 may be configured to extract the measured blood pressure from the blood pressure signal and the measured oxygen saturation from the oxygen saturation signal. The controller 115 may be configured to determine a linear correlation between the measured blood pressure and the measured oxygen saturation. The linear correlation may be based on a Pearson coefficient, for example. The Pearson coefficient may be defined as the covariance of the measured blood pressure and oxygen saturation divided by the product of their standard deviations. The result of the linear correlation may be a regression line between oxygen saturation and blood pressure, and the slope of the regression line may indicate the autoregulation status. In one possible implementation, a regression line with a negative slope (e.g., blood pressure increases after regional oxygen saturation decreases) may suggest that cerebral autoregulation is working properly while a regression line with a relatively flat or a positive slope (e.g., blood pressure remains the same or decreases after regional oxygen saturation decreases) may suggest that cerebral autoregulation is impaired. Accordingly, the controller 115 may be used to determine the autoregulation status from the linear correlation. Instead of or in addition to a regression line, the controller 115 may be configured to determine a polynomial fitting where changes in coefficient values may describe changes in the patient's cerebral autoregulation status.

The blood pressure and oxygen saturation values used to determine the linear correlation, polynomial fitting, or other relationship, may be measured over time. In one possible implementation, the values may be determined over a window from the last few minutes of measurements. The window may be any length of time, such as 4 or 5 minutes.

Once determined, the controller 115 may be configured to output the autoregulation status signal to, e.g., the output device 120, as discussed below. Moreover, the controller 115 may be configured to generate an alarm signal in certain circumstances, such as if it is determined that cerebral autoregulation is impaired. The alarm signal may therefore represent that cerebral autoregulation is impaired. The alarm signal may be transmitted with the autoregulation status signal or as a separate signal. That is, in some instances, the autoregulation status signal may only be generated if cerebral autoregulation is impaired, in which case the autoregulation status signal may also act as the alarm signal. In other instances where the autoregulation status signal is generated whether cerebral autoregulation is impaired or not, the alarm signal may be a separate signal. In any event, the alarm signal may be used, as described below, to generate a visual or audio representation of the status of cerebral autoregulation. For instance, if cerebral autoregulation is impaired, the alarm signal may cause the generation of various sounds or images designed to alert a treating physician's attention to the issue. Example visual representations may be particular colors, one or more flashing lights, the compliance value, a word description of the autoregulation status (e.g., the word “impaired”), etc. Example audio representations may include a buzzer, siren, alarm, or the like.

The output device 120 may include any device configured to receive the autoregulation status signal, the alarm signal, or both, from the controller 115 and visually and/or audibly output information in accordance with the autoregulation status signal. For instance, the output device 120 may include a display device 125 configured to provide a visual representation of the status of cerebral autoregulation determined by the controller 115. Moreover or in the alternative, the output device 120 may include an audio device 130 configured to audibly provide sounds in accordance with the alarm signal, the autoregulation status signal, or both.

In general, computing systems and/or devices, such as the controller 115 and the output device 120, may employ any of a number of computer operating systems, including, but by no means limited to, versions and/or varieties of the Microsoft Windows® operating system, the Unix operating system (e.g., the Solaris® operating system distributed by Sun Microsystems of Menlo Park, Calif.), the AIX UNIX operating system distributed by International Business Machines of Armonk, N.Y., and the Linux operating system. Examples of computing devices include, without limitation, a computer workstation, a server, a desktop, notebook, laptop, or handheld computer, or some other known computing system and/or device.

Computing devices generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of known computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

Databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners, as is known. A file system may be accessible from a computer operating system, and may include files stored in various formats. An RDBMS generally employs the Structured Query Language (SQL) in addition to a language for creating, storing, editing, and executing stored procedures, such as the PL/SQL language.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

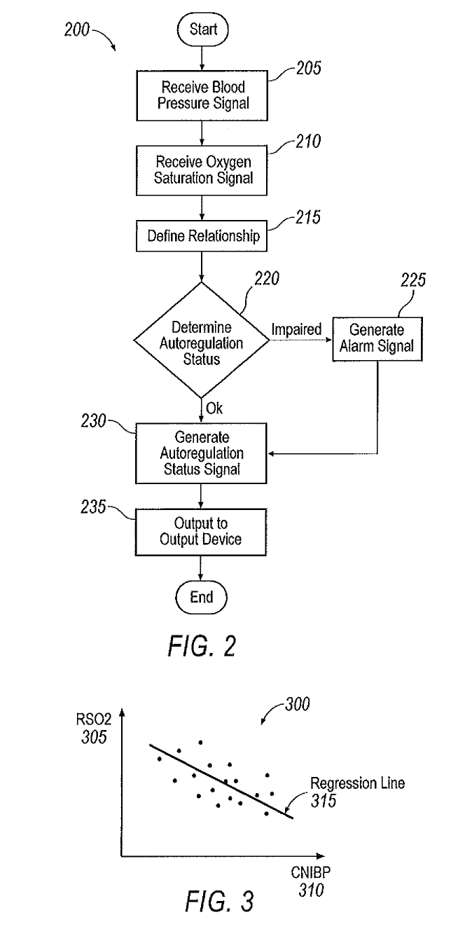

FIG. 2 illustrates a flow chart of an exemplary process 200 that may be implemented by the system 100 of FIG. 1 to, for example, non-invasively determine whether cerebral autoregulation of a patient has become impaired and to take an appropriate remedial measure.

At block 205, the controller 115 may receive a blood pressure signal from, e.g., the first sensor 105. The blood pressure signal may represent a non-invasive measure of a patient's blood pressure. The controller 115, as discussed above, is configured to determine the measured blood pressure from the blood pressure signal.

At block 210, the controller 115 may receive the oxygen saturation signal. In one exemplary approach, the oxygen saturation signal may be received from the second sensor 110, which may generate the oxygen saturation signal following a non-invasive measure of oxygen saturation. As such, the oxygen saturation signal may represent a non-invasive measure of a patient's oxygen saturation, such as regional oxygen saturation, hemoglobin, or the like. The controller 115, as discussed previously, is configured to determine the measured oxygen saturation from the oxygen saturation signal.

At block 215, the controller 115 may define a relationship between the measured blood pressure determined from the blood pressure signal and the measured oxygen saturation determined from the oxygen saturation signal. In one exemplary implementation, defining the relationship between the measured blood pressure and oxygen saturation may include determining a linear correlation, such as a Pearson coefficient, between these physiological parameters. In addition or in the alternative, the controller 115 may be configured to derive the cerebral oximetry index measurement from the linear correlation or some other relationship between the measured blood pressure and the measured oxygen saturation.

At decision block 220, the controller 115 may determine the autoregulation status. The autoregulation status may be based, at least in part, on the relationship defined at block 215. For instance, the controller 115 may determine the slope of the linear correlation defined at block 215. If the slope of the linear correlation is positive (e.g., blood pressure stays substantially the same or decreases following a drop in regional oxygen saturation), the controller 115 may determine that cerebral autoregulation is impaired, and the process 200 may continue at block 225. If the slope of the linear correlation is negative (e.g., blood pressure increases after regional oxygen saturation drops), the controller 115 may determine that cerebral autoregulation is functioning properly, and the process 200 may continue at block 230.

At block 225, the controller 115 may generate an alarm signal. The alarm signal may indicate impaired cerebral autoregulation and, upon receipt at the output device 120, cause the output device 120 to present a visual alarm, an audio alarm, or both, to a treating physician, as discussed above. In some instance, block 225 is optional, and the alarm signal may be omitted or may be included with the autoregulation status signal generated at block 230.

At block 230, the controller 115 may generate the autoregulation status signal in accordance with the determination made at block 220. For instance, if the determination at block 220 is that cerebral autoregulation is impaired, the controller 115 may generate the autoregulation status signal to represent impaired cerebral autoregulation. Alternatively, if the determination at block 220 is that cerebral autoregulation is functioning properly, the controller 115 may generate the autoregulation status signal accordingly.

At block 235, the controller 115 may output the autoregulation status signal, the alarm signal, or both, to the output device 120. Upon receipt of the autoregulation status signal, the output device 120 may present any number of visual and/or audio representations of the autoregulation status to a treating physician. In addition, the autoregulation status signal may include alarms that may be otherwise carried by the alarm signal, as discussed above. Further, the autoregulation status signal may further include the measured blood pressure, the measured oxygen saturation, or any combination of these or other physiological parameters. The output device 120 may respond to the alarm signal in the manner described above, e.g., with respect to block 225.

The process 200 may end after block 235.

FIG. 3 is an exemplary graph 300 of regional oxygen saturation 305 (e.g., the measured oxygen saturation) on the Y-axis plotted against the measured blood pressure 310 on the X-axis. The linear correlation 315 is illustrated as a regression line based on, e.g., a Pearson coefficient. The slope of the line 315 representing linear correlation is negative, which as discussed above, may indicate that cerebral autoregulation is functioning properly. The controller 115 therefore, when presented with the exemplary values of FIG. 3, may conclude that cerebral autoregulation is functioning properly and generate the autoregulation status signal accordingly. If the line 315 had a positive slope, the controller 115 may conclude the cerebral autoregulation is impaired and generate the autoregulation status signal and possibly a separate alarm signal accordingly. In some instances, the autoregulation status signal may cause a graph similar to the graph 300 of FIG. 3 to be presented on the output device 120 for presentation to a treating physician.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

Accordingly, it is to be understood that the above description is intended to be illustrative and not restrictive. Many embodiments and applications other than the examples provided would be apparent upon reading the above description. The scope of the invention should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the invention is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

1. A system comprising:
a controller configured to receive a blood pressure signal representing a non-invasive measure of blood pressure and an oxygen saturation signal representing a non-invasive measure of oxygen saturation,
wherein the controller is configured to generate an autoregulation status signal representing a status of cerebral autoregulation, and
wherein the autoregulation status signal is based at least in part on a relationship between the measured blood pressure and the measured oxygen saturation.

2. A system as set forth in claim 1, wherein the controller is configured to determine a linear correlation between the measured blood pressure and the measured oxygen saturation and determine an autoregulation status based on the linear correlation.

3. A system as set forth in claim 2, wherein the controller is configured to determine the autoregulation status based at least in part on a slope of the linear correlation.

4. A system as set forth in claim 1, wherein the controller is configured to output the autoregulation status signal to an output device.

5. A system as set forth in claim 1, wherein the controller is configured to determine the autoregulation status based at least in part on the relationship between the measured blood pressure and the measured oxygen saturation.

6. A system as set forth in claim 1, wherein the controller is configured to generate an alarm signal if the autoregulation status indicates that cerebral autoregulation is impaired.

7. A system as set forth in claim 1, wherein controller is configured to derive a cerebral oximetry index measurement from the relationship between the measured blood pressure and the measured oxygen saturation.

8. A system as set forth in claim 7, wherein the controller is configured to derive the cerebral oximetry index measurement based at least in part on a linear correlation between the measured blood pressure and the measured oxygen saturation.

9. A system as set forth in claim 1, further comprising a first sensor configured to non-invasively measure blood pressure and generate the blood pressure signal.

10. A system as set forth in claim 9, further comprising a second sensor configured to non-invasively measure oxygen saturation and generate the oxygen saturation signal.

11. A method comprising:
receiving a blood pressure signal representing a non-invasive measure of blood pressure;
receiving an oxygen saturation signal representing a non-invasive measure of oxygen saturation;
defining a relationship between the measured blood pressure and the measured oxygen saturation;
determining an autoregulation status based at least in part on the defined relationship; and
generating an autoregulation status signal representing the determined autoregulation status.

12. A method as set forth in claim 11, wherein defining the relationship between the measured blood pressure and the measured oxygen saturation includes determining a linear correlation between the measured blood pressure and the measured oxygen saturation.

13. A method as set forth in claim 12, wherein determining the autoregulation status is based at least in part on a slope of the linear correlation.

14. A method as set forth in claim 11, further comprising generating an alarm signal if the autoregulation status indicates that cerebral autoregulation is impaired.

15. A method as set forth in claim 11, further comprising deriving a cerebral oximetry index measurement from the relationship between the measured blood pressure and the measured oxygen saturation.

16. A method as set forth in claim 15, wherein deriving the cerebral oximetry index measurement includes deriving the cerebral oximetry index measurement from a linear correlation between the measured blood pressure and the measured oxygen saturation.

17. A system comprising:
a first sensor configured to non-invasively measure blood pressure and generate a blood pressure signal;
a second sensor configured to non-invasively measure oxygen saturation and generate an oxygen saturation signal;
a controller in communication with the first sensor and the second sensor and configured to receive the blood pressure signal and the oxygen saturation signal, wherein the controller is configured to generate an autoregulation status signal representing a status of cerebral autoregulation,
wherein the autoregulation status signal is based at least in part on a linear correlation between the measured blood pressure and the measured oxygen saturation, the linear correlation forming a regression line when plotted, and
wherein the controller is configured to determine the autoregulation status based at least in part on a slope of the regression line.

18. A system as set forth in claim 17, further comprising an output device configured to receive the autoregulation status signal from the controller, wherein the output device is configured to display a visual representation of the status of cerebral autoregulation.

19. A system as set forth in claim 17, wherein the controller is configured to generate an alarm signal if the autoregulation status indicates that cerebral autoregulation is impaired.

20. A system as set forth in claim 1, wherein controller is configured to derive a cerebral oximetry index measurement from the linear correlation between the measured blood pressure and the measured oxygen saturation.
